(19) United States
(12) Patent Application Publication
(10) Pub. No.: US 2012/0202260 A1
(43) Pub. Date: Aug. 9, 2012

(54) HYBRID BIOREFINING AND GASIFICATION OF LIGNOCELLULOSIC FEEDSTOCKS

(75) Inventor: John Ross Maclachlan, Burnaby (CA)

(73) Assignee: LIGNOL INNOVATIONS LTD., Burnaby, BC (CA)

(21) Appl. No.: 13/393,000

(22) PCT Filed: Sep. 3, 2010
(86) PCT No.: PCT/CA2010/001389

§ 371 Date: Apr. 25, 2012

Related U.S. Application Data
(60) Provisional application No. 61/239,926, filed on Sep. 4, 2009.

(51) Int. Cl. C12P 7/04; B01J 19/00; C01B 3/02; C07C 27/00

(52) U.S. Cl. 435/157; 518/702; 422/187; 252/373

(57) ABSTRACT

Processes and systems for concurrent recovery of lignin derivatives and syngas from a lignocellulosic feedstock. The processes and systems therefor generally comprise the steps of: (a) perfusing and cooking the lignocellulosic feedstock with a suitable organic solvent for a suitable period of time thereby producing a cellulosic solids output stream and a spent liquid solvent stream, said spent liquid solvent stream comprising solubilized lignin derivatives and other organic compounds; (b) separating said cellulosic solids output stream and said spent liquid solvent stream; (c) recovering lignin derivatives from the spent liquid solvent stream thereby producing at least a partially de-lignified spent liquid solvent stream; (d) recovering a portion of the organic solvent from the at least partially de-lignified spent liquid solvent stream thereby producing a stillage; and (e) gasifying the cellulosic solids output stream thereby producing a combustible syngas.

### FIELD OF THE INVENTION

This invention relates to systems and methods for recovery of lignin derivatives from plant biomass. More particularly, this invention relates to concurrent recovery of lignin derivatives and combustible fuels from lignocellulosic feedstocks.

### BACKGROUND OF THE INVENTION

Significantly increasing world-wide demands for petroleum-based fuels combined with global concerns over crude oil pricing instability/volatility and uncertainty of reliable long-term supplies have resulted in considerable efforts placed on the development of alternative sources of combustible fuels. Fuel alcohol produced from plant materials is considered a primary candidate for supplementing and/or replacing petroleum-based fuels. Fuel alcohol production from fermentation of grains has a long commercial history but is considered a less-than ideal alternative because its production diverts grains from use in foodstuffs. Fuel alcohol production from sugar cane syrup is also well-established but its supply is limited. Furthermore, alcohol production from sugar cane is accompanied by significant accumulations of solid wastes in the form of bagasse which are typically disposed of by burning or are stockpiled for slow decomposition. For these reasons, fuel alcohol production from processing of lignocellulosic feedstocks is considered a preferred approach. Current commercial development and scale-up of cellulosic fuel alcohol production generally follows one of two strategies.

The first strategy involves processing, commonly referred as pulping, of lignocellulosic feedstocks to produce cellulosic solids that are subsequently separated from concurrently produced liquid streams. The liquid streams, also referred to as liquors, generally comprise biomass components that have been solubilized or otherwise separated from cellulosic solids during the pulping process. The recovered cellulosic solids i.e. pulps, are chemically or enzymatically hydrolyzed to produce monosaccharides that are subsequently or concurrently fermented to produce beers comprising short-chain alcohols such as methanol, ethanol, and butanol. The beers are recovered from the fermentation systems, then distilled and refined to produce fuel-grade alcohols. Cellulosic pulps suitable for hydrolysis and fermentation production of alcohols can be produced with a variety of well-known mechanical and chemical procedures that are exemplified by processes based on kraft-pulping, steam-explosion, thermo-mechanical disruption, strong or weak acids, strong or weak bases, and organic solvents. Regardless of pulping method, a common problem associated with cellulosic alcohol production is the retention of lignin components in cellulosic pulps after pulping has been completed. Native lignins are materials produced by plants during their normal growth to cement cellulose fibers together thereby providing plants with their structural integrity and support. Native lignins are typically fragmented and at least partially dissolved into moieties during pulping. Lignin fragments remaining in pulp are known to significantly interfere with enzymatic hydrolysis of cellulose, and their presence adds considerably to the cost of production of cellulosic alcohol fuels.

The liquid streams recovered from pulping of lignocellulosic feedstocks typically comprise solubilized plant components including hemicellulose sugars, polysaccharides, oligosaccharides, lignin moieties (also referred to as derivatives) and other organic compounds. When inorganic and/or organic solvents are used in the pulping processes, the liquors may also contain organic compounds such as ketones, aldehydes, carboxylic acids, furfurals, acetic acids, phenolic compounds and terpenoids among others. Most of the solubilized plant components present in post-pulping liquors have some commercial value and therefore, most cellulosic alcohol production schemes typically include some liquor processing steps to recover some of these components. However, problems commonly encountered include excessive variability in recovery efficiency of selected components and the complex undefined nature of the recovered components. More development work is required to optimize components recovery from liquors and their subsequent refining.

The second strategy for cellulosic fuel alcohol production generally involves gasification of lignocellulosic feedstocks at high temperatures with air or oxygen and steam inputs to produce gas mixtures comprising carbon monoxide (CO) and hydrogen (H₂). These gas mixtures are commonly referred to as syngas. Syngas is directly combustible as a fuel or alternatively, may be converted using known methods into short-chain alcohols and/or chemicals such as alkanes, olefins and oxygenates among others. Alcohols produced from syngas can be refined into fuel-grade alcohols. Depending on the nature of the lignocellulosic feedstock and the gasification system used, syngas is concurrently produced with a solid waste material called ash or slag that comprises minerals and/or metals. However, problems associated with syngas production from lignocellulosic feedstocks include fouling caused by the non-carbon elements from plant biomass, and/or their accumulation in the solid residues which then become a disposal issue.

### SUMMARY OF THE INVENTION

Recent work on development of processing strategies for converting lignocellulosic feedstocks into cellulosic alcohols, demonstrated that pulping of a wide range of lignocellulosic feedstocks with organic solvents, also referred to as organosolv pretreatment or pulping, generally results in the solubilization of unique classes of lignin derivatives that may be separated and sequentially recovered from the spent solvent black liquor streams. These lignin derivatives may be characterized by their physico-chemical and functional properties, and appear to be useful as feedstocks and/or alternatively, for incorporation into a wide variety of industrial chemical manufacturing processes. Furthermore, it is apparent that the types of lignin derivatives solubilized during organosolv pulping can be manipulated and tailored by adjusting various physico-chemical components of the pulping process conditions. Accordingly, organosolv biorefining of lignocellulosic feedstocks may have significant commercial value associated solely with the recovery and processing of novel lignin derivatives, in which case, the cellulosic solids streams separated during organosolv pulping could be considered as waste streams to be disposed of efficiently, cost-effectively, and using environmentally acceptable methods.

Cellulosic solids waste streams produced during commercial-scale organosolv biorefining of lignocellulosic feedstocks for recovery of lignin derivatives only, could be disposed of by burning, e.g., to produce heat and energy for plant operations. However, it is apparent that because organosolv pulping solubilizes and removes up to about 95% or more of the cementing lignin materials comprising lignocellulosic biomass commonly used as biorefining feedstocks, extracellular organic compounds which comprise most plant biomass mineral and metal constituents are also solubilized or suspended in the black liquor stream and are subsequently separated from the cellulosic solids. Consequently, organosolv-produced cellulosic solids would contain substantially lower (i.e., trace amounts) minerals and metals than feedstocks processed with other pulping processes e.g., kraft and sulphite. Therefore, gasification of organosolv-produced cellulosic solids should significantly reduce the problems commonly associated with slag or ash production during gasification of unprocessed lignocellulosic feedstocks.

Accordingly, exemplary embodiments of the present invention relate to systems, processes and equipment configurations for organosolv biorefining of lignocellulosic feedstocks for concurrent recovery of lignin derivatives from spent solvent liquid streams and gasification of cellulosic solids streams. De-lignified or alternatively partially de-lignified liquid streams may be concurrently gasified with the cellulosic solids streams.

Some exemplary embodiments relate to integrated biorefining systems and methods for receiving, processing and organosolv pulping of lignocellulosic feedstocks thereby producing spent solvent black liquor streams and cellulosic solids streams. The black liquor streams are separated from the cellulosic solids and are recovered from organosolv pulping components and are de-lignified to recover at least one class of lignin derivatives. It is optional to recover two more classes lignin derivatives by selectively incorporating into the systems and methods, sequential lignin separation and recovery steps. Exemplary lignin separation steps include temperature flashing i.e., rapidly reducing the temperature of black liquor streams upon their egress from organosolv pulping components, rapid depressurization of black liquor stream, rapid dilution of black liquor streams with water, each of which causes some lignin derivatives to precipitate from black liquor solutions. The separated cellulosic solids are recovered from organosolv pulping components and are gasified to produce combustible syngas. Some aspects relate to recovery of delignified black liquor streams and their concurrent gasification with the cellulosic solids.

Some aspects relate to recovery of a portion of the spent organic solvent from de-lignified black liquor streams. The recovered organic solvent may be recycled back into the organosolv pulping components. Stillage produced during recovery of organic solvent may be concurrently gasified with the cellulosic solids. Some aspects relate to further processing of the stillage for recovery of other solubilized and/or suspended plant components exemplified by furfurals, hemicelluloses and hemicellulose breakdown products, monosaccharides, oligosaccharides, polysaccharides, acetic acid, lipophylic extractives, phenolic compounds, terpenoids.

Some aspects relate to recovery and further processing of a portion of syngas to produce therefrom short-chain alcohols exemplified by methanol, ethanol, and butanol. Such short-chain alcohols may be refined to produce fuel-grade alcohols.

Some aspects relate to recovery and further processing by Fischer-Tropsch reactions, of a portion of syngas to produce therefrom chemicals useful for industrial applications. Such chemicals are exemplified by alkanes, olefins, oxygenates, biodiesel, and the like.

Some exemplary embodiments relate to modular biorefining systems and methods for receiving, processing and organosolv pulping of lignocellulosic feedstocks for concurrent recovery of lignin derivatives from spent solvent black liquor streams and gasification of cellulosic solids streams to produce combustible syngas.

Suitable modular biorefining processing systems of the present invention comprise at least:

a first module comprising a plurality of equipment configured for: (a) receiving and processing lignocellulosic fibrous feedstocks, then (b) pulping under controlled temperature and pressure conditions the processed feedstocks with suitable solvents configured for physico-chemically disrupting the lignocellulosic feedstock into a solids fraction comprising mostly cellulosic pulps and a black liquor liquids fraction comprising spent solvents containing therein lignins, lignin-containing compounds, monosaccharides, oligosaccharides and polysaccharides, dissolved and suspended solids comprising hemicelluloses and celluloses and other organic compounds, and (c) providing a first output stream comprising the black liquor liquids fraction and a second output stream comprising a black liquor liquids fraction comprising the cellulosic solids fraction;

a second processing module configured for separating lignins from the black liquor liquids fraction thereby producing at least a partially de-lignified liquid fraction. It is optional to recover two more classes of lignin derivatives by selectively incorporating into the systems and methods, sequential lignin separation and recovery steps. It is optional to recover a portion of spent organic solvent from the de-lignified liquid fraction thereby producing at least a partially de-lignified stillage. The recovered organic solvent may be recycled back to the first module for organosolv pulping of fresh lignocellulosic feedstock. It is also optional to recover from the at least partially de-lignified liquid fraction or from the partially de-lignified stillage, one or more of furfurals, hemicelluloses and hemicellulose breakdown products, monosaccharides, oligosaccharides, polysaccharides, acetic acid, lipophylic extractives, phenolic compounds, terpenoids, and the like, thereby producing at least a partially de-lignified liquid waste stream. It is optional to configure the second module for transfer of portions of the partially de-lignified liquid fraction and/or the partially de-lignified stillage and/or the partially de-lignified liquid waste stream to the third processing module for gasification;

a third processing module configured for receiving and gasifying the cellulosic solids fraction thereby producing a combustible syngas. It is optional to further configure the third processing module for recovery of a portion of the syngas for further processing to produce therefrom one or more short-chain alcohols exemplified by methanol, ethanol and butanol. Such short-chain alcohols are suitable for refining into fuel-grade alcohols. It is also optional to further configure the third processing module for recovery of a portion of the syngas for further processing by Fischer-Tropsch reactions to produce therefrom one or more chemical streams useful for production of alkanes, olefins, oxygenates, biodiesel, and the like.

Some aspects relate to incorporation of an optional fourth processing module configured to receive at least a portion of the cellulosic solids fraction from the first processing module, and further process the cellulosic solids to produce one or more of highly-purified crystalline cellulose and sugar syrups. The optional fourth processing module may be additionally configured for fermenting sugar syrups produced from the cellulosic solids, into short-chain alcohols.

The black liquor streams are separated from the cellulosic solids and are recovered from organosolv pulping components and are at least partially de-lignified to recover at least one class of lignin derivatives. It is optional to recover two more classes of lignin derivatives by selectively incorporating into the systems and methods, sequential lignin separation and recovery steps. Exemplary lignin separation steps include temperature flashing i.e., rapidly reducing the temperature of black liquor streams upon their egress from organosolv pulping components, rapid depressurization of black liquor stream, rapid dilution of black liquor streams with water, each of which causes some lignin derivatives to precipitate from black liquor solutions. It is within the scope of the present inventions to recover additional lignins from the cellulosic solids stream by washing one or more times with a suitable solvent prior to the gasification step. The separated cellulosic solids are recovered from organosolv pulping components and are gasified to produce combustible syngas. Suitable solvents are exemplified by water, diluted organic acids and diluted inorganic acids. Some aspects relate to recovery of delignified black liquor streams and their concurrent gasification with the cellulosic solids.

### BRIEF DESCRIPTION OF THE DRAWINGS

The present invention will be described in conjunction with reference to the following drawings in which:

FIG. 1 is a schematic flowchart of an exemplary embodiment of the present invention of a hybrid system for organosolv processing of a lignocellulosic feedstock wherein a solids output stream is gasified to produce syngas, and a liquid output stream is processed to recover lignin derivatives, to recycle a portion of the organic solvent, and to gasify the remaining liquid outputs;

FIG. 2 is an expanded schematic flowchart of the system from FIG. 1 wherein additional components are recovered from the liquid output stream;

FIG. 3 is an expanded schematic flowchart of the system from FIG. 1 wherein the syngas is purified and further processed to produce short-chain alcohols by catalytic conversion;

FIG. 4 is an expanded schematic flowchart of the system from FIG. 1 wherein the syngas is purified and further processed to produce short-chain alcohols by fermentation;

FIG. 5 is an expanded schematic flowchart of the system from FIG. 1 wherein the syngas is purified and further processed as a feedstock for production of industrial chemicals;

FIG. 6 is an expanded schematic flowchart of the system from FIG. 1 wherein the syngas is purified and further processed to concurrently produce short-chain alcohols and industrial chemicals;

FIG. 7 is an expanded schematic flowchart of the system from FIG. 1 wherein additional components are recovered from the liquid output stream concurrently while syngas produced from the solids output stream is purified and further processed as a feedstock for concurrent production of short-chain alcohols and industrial chemicals; and

FIG. 8 is a schematic flowchart of the system from FIG. 1 showing a modular configuration for organosolv pulping of a lignocellulosic feedstock in a first module to produce a liquid output stream and a cellulosics solids stream, a second module wherein lignin derivatives and additional components are recovered from the liquid output stream, and a third module for gasification of the cellulosic solids stream to produce syngas may be subsequently purified and processed into alcohols and chemical feedstocks.

### DETAILED DESCRIPTION OF THE INVENTION

Exemplary embodiments of the present invention relate to processes and systems configured for separating lignocellulosic feedstocks into two output streams wherein one stream is a liquid stream comprising solubilized lignin moieties and lignin derivatives, and the other stream comprises cellulosic solids that are subsequently gasified. Some exemplary embodiments relate to recovery of lignin derivatives from the liquid stream. Some exemplary embodiments relate to recovery of syngas from gasification of the cellulosic solids.

Suitable lignocellulosic feedstocks are exemplified by angiosperm fibrous biomass, gymnosperm fibrous biomass, annual and perennial field crop fibrous biomass, fruit and vegetable pulps, bagasse, waste paper and wood materials, the like, and mixtures thereof.

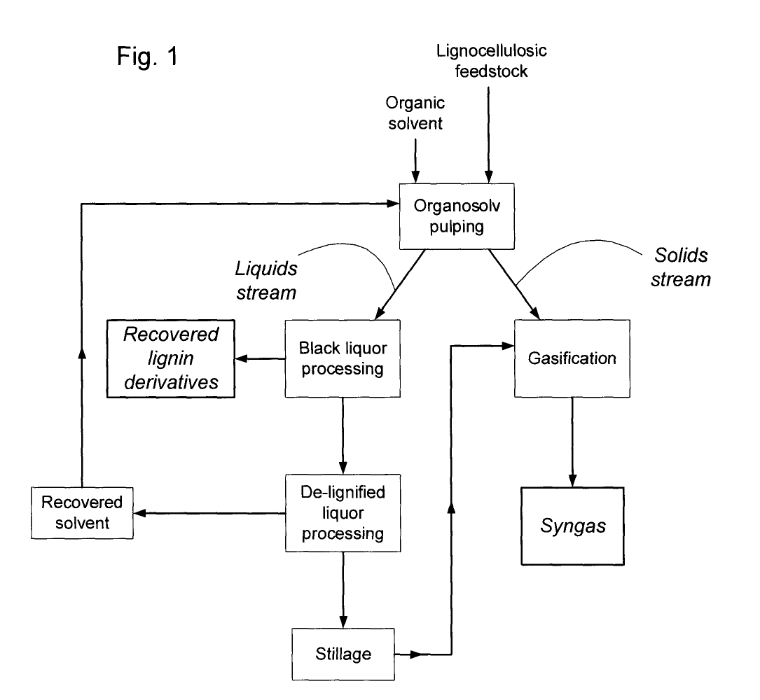

One exemplary embodiment of the present invention is shown in FIG. 1 and generally relates to processes and systems for separating lignocellulosic feedstocks into solids streams comprising cellulosic components and liquid streams comprising solubilized and/or suspended lignin derivatives and other plant components. Suitable processes and processing systems are exemplified by biorefining, pulping processes and systems based on perfusing, commingling and cooking lignocellulosic feedstocks in organic solvents at suitably elevated temperatures and pressures, thereby producing solid and semi-solid amorphous cellulosic pulp materials and liquors comprising spent solvents in which are solubilized and suspended plant components. It is suitable for the lignocellulosic feedstocks to be physically disrupted and/or comminuted prior to the pulping process. Suitable organic solvents are exemplified by about 35% to about 75% ethanol diluted in water. The organic solvent may be supplemented with an inorganic acid or alternatively, an organic acid provided as a reaction catalyst. An exemplary inorganic acid is sulfuric acid. The amorphous pulp materials thus produced primarily comprise purified cellulose-rich fibers that are low in residual lignin and in which the cellulose crystallinity has been significantly reduced. The spent solvents are commonly referred to as black liquors, and typically comprise solubilized lignin moieties and lignin derivatives, furfurals, hemicelluloses and hemicellulose breakdown products, monosaccharides, oligosaccharides, polysaccharides, acetic acid, lipophylic extractives, phenolic compounds, terpenoids and spent ethanol. The solid amorphous pulp materials and black liquors are then separated into a solids output stream and a liquid stream, i.e., a black liquor stream.

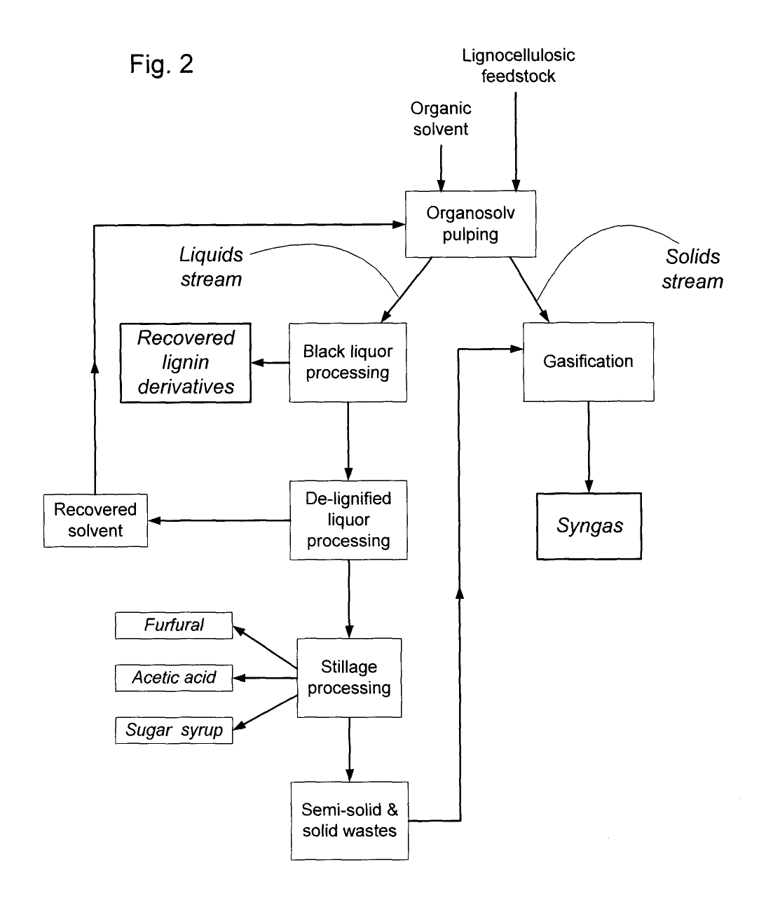

The black liquor stream is further processed to separate and remove at least some of the lignin derivatives by first flashing the liquor stream to atmospheric pressure and then rapidly diluting the liquor with water thereby causing the lignin derivatives to precipitate out of solution. The lignins are then recovered and removed for further purification and/or processing using equipment and processes known to those skilled in these arts. It is optional to recover a first class of lignin derivatives that precipitate during the flashing step prior to diluting the liquor with water. It is then possible to separate a second class of lignin derivatives that precipitate during and after the water dilution step. The at least partially de-lignified liquor stream may then be distilled to recover the ethanol solvent for reuse in pulping fresh incoming lignocellulosic feedstocks. The remaining stillage is recovered for gasification. Alternatively, as shown in FIG. 2, the stillage stream may be further processed to recover other extracted plant components such as furfurals, sugar syrups, acetic acid, phenolic compounds and terpenoids among others. The remaining waste stream may be recovered for gasification.

As shown in FIGS. 1 and 2, the cellulosic solids stream is gasified along with stillage waste streams recovered from liquor processing, to produce syngas using conventional gasification equipment, systems and processes known to those skilled in these arts. Suitable gasification systems are exemplified by counter-current up-draft gasification systems, co-current down-draft gasification systems, fluidized-bed gasification systems, entrained-flow gasification systems, multi-step thermochemical gasification systems, and the like. The syngas output from gasification of the cellulosic solids and the waste stream recovered after black liquor processing, may then be combusted to produce energy that is useful for providing power to utilities required to operate the lignocellulosic processing facility.

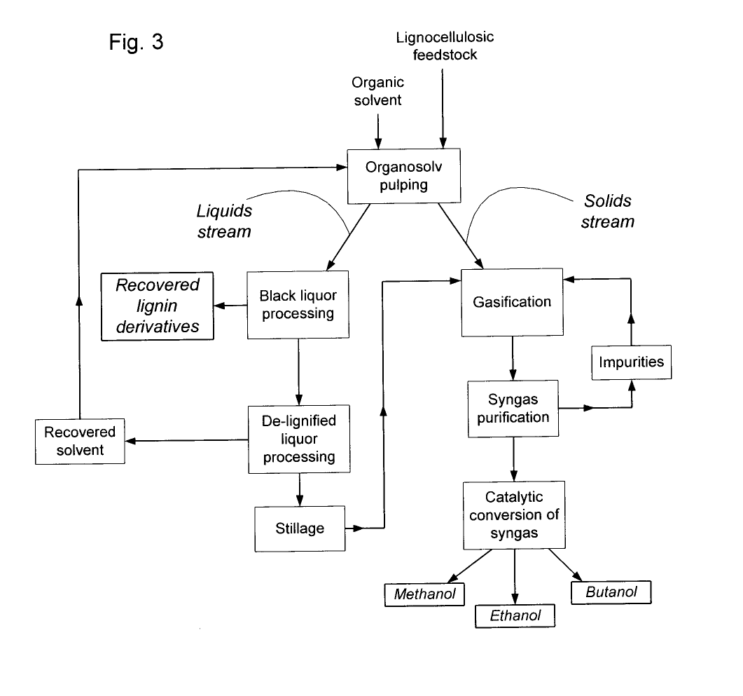

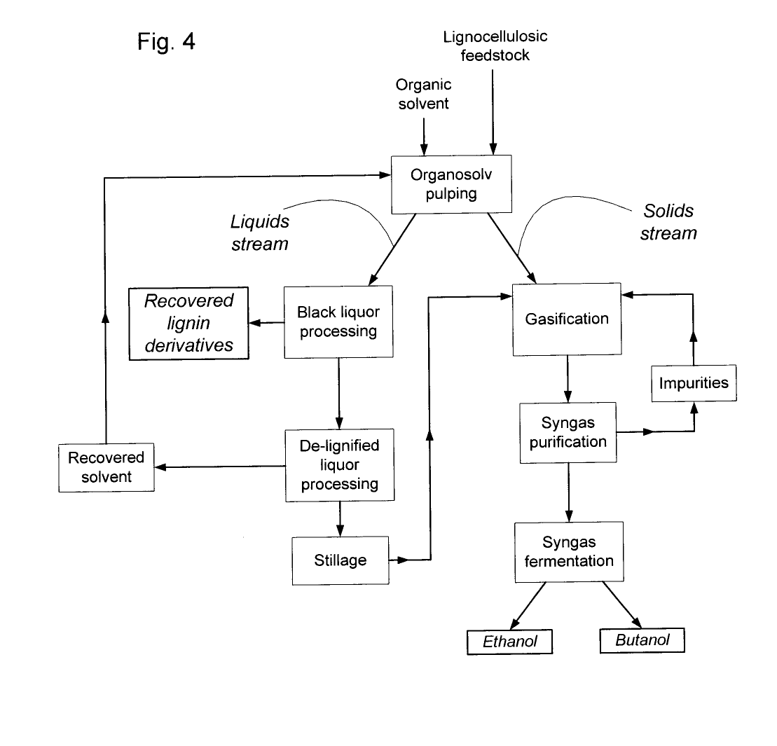

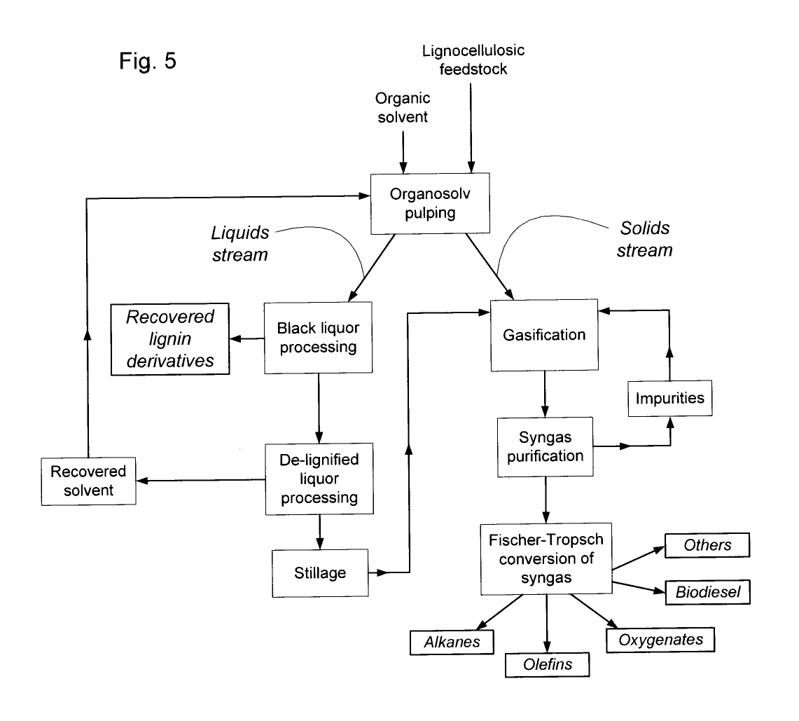

Because the mineral and metal components of lignocellulosic feedstocks are generally removed from cellulosic solids during pulping with organic solvents, syngas produced by gasification of the recovered cellulosic solids streams are generally purer than those produced by direct gasification of lignocellulosic materials. Accordingly, this syngas is particularly suitable for conversion to short-chain alcohols (FIG. 3) by reactions with selected catalysts known to those skilled in these arts or alternatively, by fermentation with ethanolagens or butanolagens (FIG. 4). Alternatively, this syngas may be converted into a variety of chemical streams including alkanes, olefins, oxygenates, biodiesel and others by application of the Fischer-Tropsch process known to those skilled in these arts (FIG. 5).

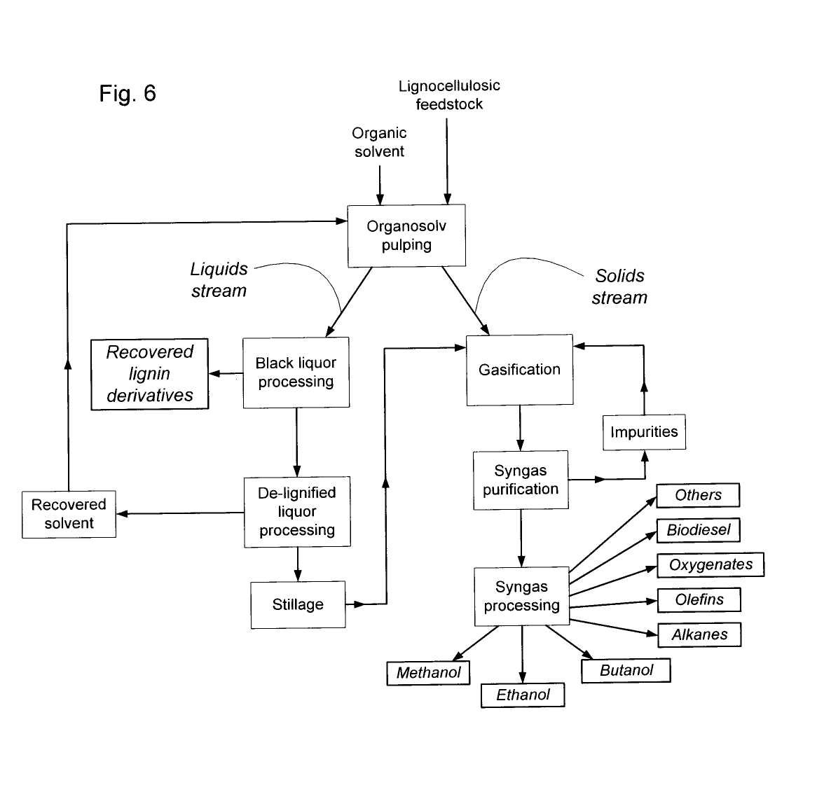

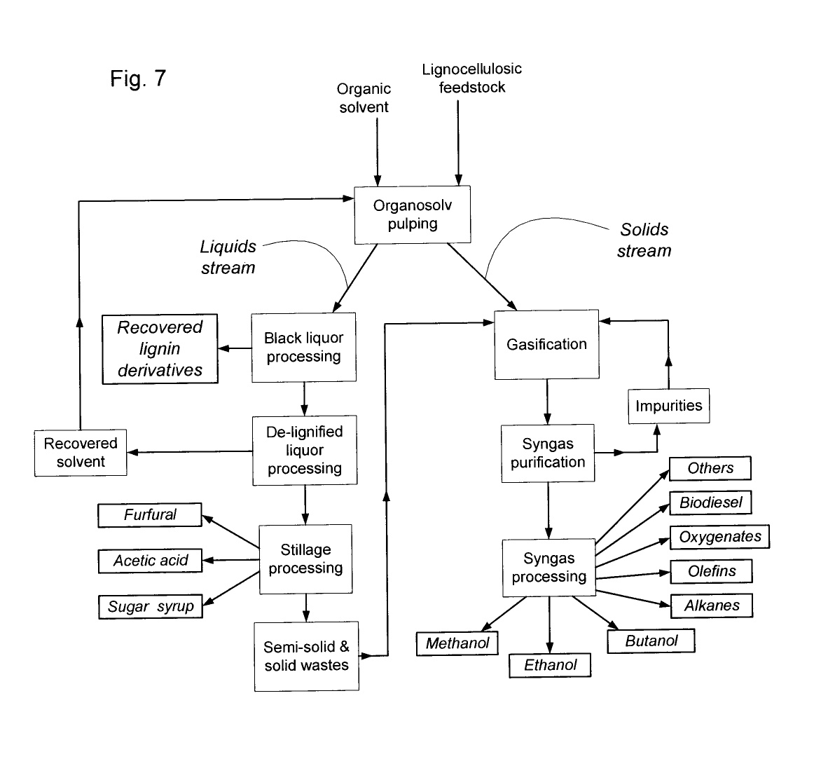

The scope of the present invention envisions the recovery of lignin derivatives from black liquor streams recovered from organosolv pulping of lignocellulosic feedstocks, recovery and recycling of at least a portion of the organic solvent and gasification of the remaining stillage concurrent with gasification of the cellulosic solids recovered from the organosolv pulping process followed by concurrent conversion of at least a portion of the syngas into shortchain alcohols and other chemical streams (FIG. 6). It is also within the scope of the present invention to further concurrently recover chemical components from the de-lignified liquor stream prior to gasification of the stillage (FIG. 7).

Those skilled in these will understand the hybrid organosolv pulping and gasification processes described herein can be carried out in processing facilities designed as integrated biorefinery systems as exemplified in FIGS. 1-7. However, it is also suitable to subdivide these integrated biorefinery systems into smaller parts (i.e., modules or components) that are interconnected but are configured such that each module can be independently created, and separately and controllably operated. However, the interconnections between the modules enable controllably directed delivery of process inputs into the individual modules, and the controllable egress and transfer of process outputs from the individual modules to other modules. For clarity, each module is configured to receive and process therein at least one input feedstock thereby producing at least two or more product output streams. Each product output stream from one module may be transferred to a second module as an input feedstock wherein it is converted into two or more new product output streams. The configurations of each module and the interconnections between the modules enable targeted and controllable recovery of some or all of each product output stream from a selected module, or alternatively, transfer of some or all of each product output stream from the selected module to another module, thereby enabling the modular biorefinery to drive different but integrated functionalities. The exemplary modular biorefineries of the present invention are characterized by functional partitioning into discrete scalable, reusable modules consisting of isolated, self-contained functional elements, rigorous use of well-defined modular interfaces including object-oriented descriptions of module functionality, ease of change to achieve technology transparency and, make use of industry standards for key interfaces. In addition to flexibility in design, modularization of biorefining systems enables incorporation of additional modules to further process product output streams thereby creating addition product capture opportunities, and also enables by-passing one or more processing modules if so desired.

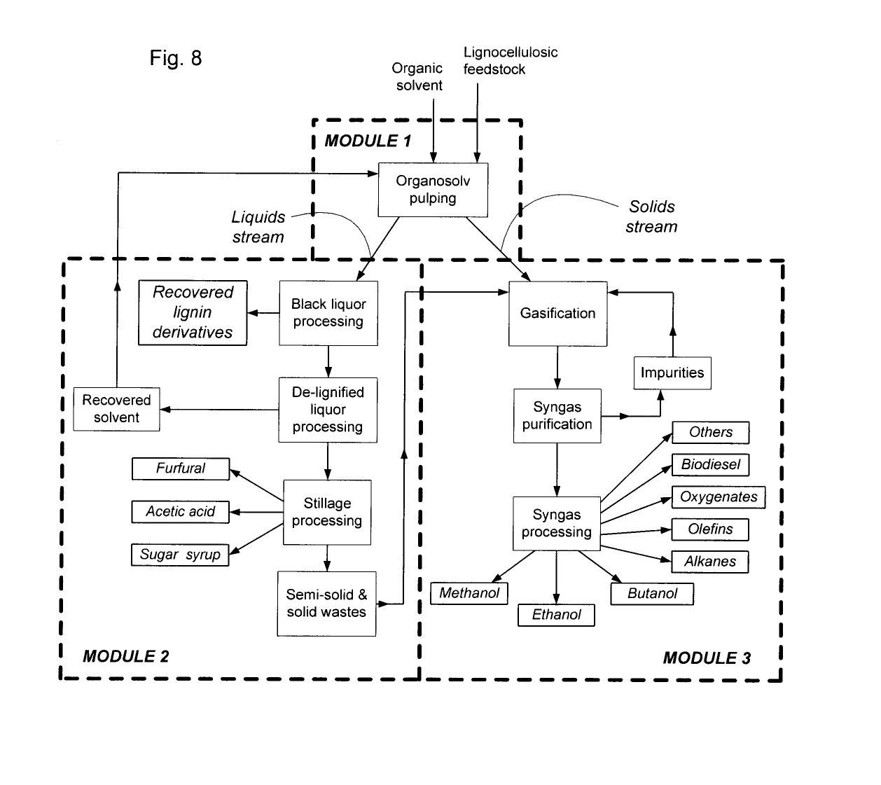

As shown in FIG. 8, it is suitable to separate the hybrid pulping-gasification system of the present invention into at least three modules wherein the first module comprises an organosolv pulping system configured for receiving and processing a lignocellulosic feedstock to produce a cellulosic solids stream and a black liquor stream comprising solubilized and/or suspending plant components, the second module comprises black liquor processing equipment configured for recovery of at least one class of lignin derivatives and a portion of the organic solvent, and the third module comprises gasification equipment configured for receiving and processing the cellulosic solids stream from the first module, and the waste stream from the second stream. The gasification equipment in the third module may be configured to cooperate with syngas recovery and processing equipment. The exemplary modular hybrid pulping-gasification system shown in FIG. 8 is amenable to communication and cooperation with additional modules. For example, a fourth module (not shown) may be interconnected with the first module for receiving and recovering a portion or all of the cellulosic solids stream for further refining into high-quality cellulose pulps and/or alternatively, for hydrolytic conversion into sugar streams that may be recovered or optionally, fermented.

Therefore, in view of numerous changes and variations that will be apparent to persons skilled in these arts, the scope of the present invention is to be considered limited solely by the appended claims.

What is claimed is:

1. A process for concurrent recovery of lignin derivatives and syngas from a lignocellulosic feedstock, the process comprising the steps of:
perfusing and cooking the lignocellulosic feedstock with an organic solvent for a suitable period of time thereby producing a cellulosic solids output stream and a spent liquid solvent stream, said spent liquid solvent stream comprising solubilized lignin derivatives and other organic compounds;
separating said cellulosic solids output stream and said spent liquid solvent stream;
recovering lignin derivatives from the spent liquid solvent stream thereby producing at least a partially de-lignified spent liquid solvent stream;
recovering a portion of the organic solvent from the at least partially de-lignified spent liquid solvent stream thereby producing a stillage; and
gasifying the cellulosic solids output stream thereby producing a combustible syngas.

2. A process according to claim 1, wherein the stillage is concurrently gasified with the cellulosic solids output stream.

3. A process according to claim 1, wherein the at least partially de-lignified solids output stream is further processed to remove therefrom at least one furfurals, acetic acid, sugar syrups, phenolic compounds and terpenoids, thereby producing a processed stillage waste stream.

4. A process according to claim 3, wherein the processed stillage waste stream is concurrently gasified with the cellulosic solids output stream.

5. A process according to claim 1, wherein the combustible syngas is further processed to recover therefrom a short-chain alcohol.

6. A process according to claim 5, wherein the short-chain alcohol is one of methanol, ethanol, and butanol.

7. A process according to claim 5, wherein further processing of the combustible syngas is one of a chemical reaction and a fermentation.

8. A process according to claim 7, wherein said chemical reaction comprises at least one reaction step of contacting the syngas with a suitable catalyst.

9. A process according to claim 1, wherein the combustible syngas is further processed to recover therefrom at least one of alkanes, olefins, oxygenates and biodiesel.

10. A process according to claim 9, wherein the further processing comprises at least one Fischer-Tropsch reaction.

11. A biorefining system for recovering lignin derivatives from lignocellulosic feedstocks, the system comprising:
a pulping unit configured to receive and process a lignocellulosic feedstock with an organic solvent to separate therefrom a cellulosic solids stream and a spent solvent black liquor stream comprising solubilized plant components;
a black liquor processing unit configured to receive and process the black liquor stream for separation and recovery therefrom of a plurality of lignin derivatives, a portion of the organic solvent, and at least a partially de-lignified stillage; and
a gasification unit configured to receive and gasify the cellulosic solids stream and to recover therefrom a syngas stream.

12. A biorefining system according to claim 11, additionally configured to recycle the recovered portion of the organic solvent into the pulping unit.

13. A biorefining system according to claim 11, wherein the black liquor processing unit is additionally configured for recovery from the de-lignified stillage of at least one of furfurals, acetic acid, sugar syrups, phenolic compounds and terpenoids, thereby producing a de-lignified stillage waste stream.

14. A biorefining system according to claim 11, additionally configured to recycle the recovered portion of the de-lignified stillage into the gasification unit.

15. A biorefining system according to claim 13, additionally configured to recycle the recovered portion of the e-lignified stillage waste stream into the gasification unit.

16. A biorefining system according to claim 11, wherein the gasification unit is additionally configured to convert a portion of the syngas stream into a short-chain alcohol.

17. A biorefining system according to claim 16, wherein the gasification unit is configured for purification of the syngas stream.

18. A biorefining system according to claim 16, wherein the gasification unit is configured for conversion of the syngas stream into a short-chain alcohol by at least one catalyzed chemical reaction.

18. A biorefining system according to claim 16, wherein the gasification unit is configured for conversion of the syngas stream into a short-chain alcohol by a fermentation process.

16. A biorefining system according to claim 11, wherein the gasification unit is additionally configured to convert a portion of the syngas stream into one of alkanes, olefins, oxygenates and biodiesel by a Fischer-Tropsch reaction.

17. A biorefining system according to claim 11, wherein the gasification unit is configured for concurrent conversion of a portion of the syngas stream into a short-chain alcohol and one of lkanes, olefins, oxygenates and biodiesel.

18. A modular biorefining system for recovering lignin derivatives from lignocellulosic feedstocks, the system comprising:
a first module comprising a pulping unit configured to receive and process a lignocellulosic feedstock with an organic solvent to separate therefrom a cellulosic solids stream and a black liquor stream comprising solubilized plant components;
a second module comprising a black liquor processing unit configured to receive and process the black liquor stream to separate and recover therefrom a plurality of lignin derivatives, a portion of the organic solvent, and a de-lignified stillage; and
a third module comprising a gasification unit configured to receive and gasify the cellulosic solids stream and to recover therefrom a syngas stream.

19. A modular biorefining system according to claim 18, additionally provided with a fourth module configured for receiving and processing therein a portion of the cellulosic solids stream from the first module.
